(19) United States
(12) Patent Application Publication
(10) Pub. No.: US 2025/0072732 A1
(43) Pub. Date: Mar. 6, 2025

(54) HANDLE FOR AN ENDOSCOPE

(71) Applicant: AMBU A/S, Ballerup (DK)

(72) Inventors: Anders KLÜVER, Ballerup (DK); Thomas Bachgaard JENSEN, Værløse (DK); Alistair David MORTON, Copenhagen (DK)

(73) Assignee: AMBU A/S, Ballerup (DK)

(21) Appl. No.: 18/824,836

(22) Filed: Sep. 4, 2024

(30) Foreign Application Priority Data
Sep. 5, 2023 (EP) 23195488.4
Sep. 5, 2023 (EP) 23195491.8
Feb. 28, 2024 (EP) 24160240.8
Aug. 29, 2024 (EP) 24197365.0

(51) Int. Cl. A61B 1/005

(52) U.S. Cl. CPC A61B 1/0053

(57) ABSTRACT

An endoscope including a handle assembled by application of fasteners being integral with a housing shell, the assembled housing shells forming a compartment. The handle includes at least one component selected from the group including: a roller for bending a bending section, a wire pipe fastener connecting wire pipes to a handle shell, and an encasement for a printed circuit board. The handle includes interfaces configured to secure the at least one component to a housing shell inside the compartment, the interfaces being integral with a housing shell. The housing shells are made from polypropylene.

### CROSS-REFERENCE TO RELATED APPLICATIONS

This application claims priority from and the benefit of European Patent Application No. 23 195 488.4 and European Patent Application No. 23 195 491.8, both filed Sep. 5, 2023, European Patent Application No. 24 160 240.8, filed Feb. 28, 2024, and European Patent Application No. 24 197 365.0, filed Aug. 29, 2024; the disclosures of said applications are incorporated by reference herein in their entirety.

### TECHNICAL FIELD

The present disclosure relates to an endoscope comprising a handle including handle shells.

### BACKGROUND

Single-use endoscopes mitigate or eliminate the risk for cross-contamination and improve availability of such instruments where and when they are needed in addition to eliminating the time and cost involved to clean re-usable endoscopes. To reduced cost, single-use endoscopes are often made with a relatively high polymer content and are assembled using adhesives.

Single-use endoscopes are often made with a relatively high content of polymers and assembled by use of gluing processes. This keeps manufacturing costs low.

Studies have shown that the carbon footprint of maintaining and cleaning re-usable endoscopes may be equal to or greater than the carbon footprint of single-use endoscopes. The increasing concern for environmental impact and climate change has resulted in a focus on reducing the carbon dioxide footprint from single-use products. Designing single-use devices with lower carbon footprint materials comes with drawbacks, however, since such materials may have different characteristics in a range of variables including temperature, adhesion, shrinking, warpage, rigidity, and strength.

### SUMMARY

It is an object of the present disclosure to provide a single-use endoscope with even lower carbon footprint than prior single-use endoscopes. The carbon footprint may be reduced by selecting lower carbon footprint materials and reducing the use of adhesives to bond plastic components. Synergistically, by configuring components in novel ways the reduction of the carbon footprint may result in lower material and assembly costs.

In a first aspect, the disclosure provides an endoscope comprising a handle and an insertion cord with a bending section which is bendable by manipulation of steering wires movably arranged in wire pipes.

In one embodiment according to the first aspect, a handle is assembled from handle shells by fasteners integrated with the handle shells. The handle shells comprise, or consist essentially of, polypropylene (PP), which may be virgin, recycled, bio-based and/or carbon based. The handle shells may be affixed to each other with the especially configured fasteners to avoid using adhesives. As used herein, “integrated” means made together in a one-piece part, for example by injection molding the handle shells with the especially configured fasteners. Each of the fasteners is made of the same material and in one-piece with one or the other of the two housing shells. The assembled housing shells form housing with a compartment. The handle also comprises at least one component and an interface configured to secure the at least one component to a housing shell inside the compartment. Part of the interface is made in one-piece with, and of the same material as, a housing shell. The interface extends from the housing shell into the compartment.

Handles (and other components of single-use endoscopes) are typically manufactured from acrylonitrile butadiene styrene (ABS). ABS is an amorphous polymer suitable for applications that require high strength, stiffness, and heat resistance, while PP, a semi-crystalline polymer, is more suitable for applications that require high flexibility. For example, the elongation at break of ABS is about 30% while the elongation at break of PP may be about 100%. Further, ABS requires more heat to maintain the polymer at a high level while feeding the material to an injection molding machine. Otherwise, the material will cool quickly which can result in structural flaws in the final product. And ABS, due to being amorphous, does not lend itself well to on-the-fly processing changes during manufacturing, which changes result in more scrapped material relative to PP. In sum, an endoscope according to the present disclosure reduces carbon footprint by using PP instead of ABS and provides novel features especially configured to solve the problems caused by such substitution, including lower rigidity and lower adhesion characteristics. The carbon footprint can be reduced further by using bio-PP instead of carbon based PP.

In this disclosure, bio-based PP is understood to originate from biomass, which is renewable, e.g., within a time span of up to 10 years or up to 50 years. This is opposed to so-called synthetic PP which is made from a fossil-based feedstock, such as crude oil, which is not renewable, at least not when considering a time span of up to e.g., a thousand years. One way to establish whether the PP is bio-based or based on a fossil feedstock, is by a carbon 14 analysis. A carbon 14 analysis can also provide an estimate of the relationship between the content of bio-based PP and the content of fossil-based PP.

Bio-based polypropylene may be manufactured from natural materials such as lignocellulosic biomass (starch and cellulose), fatty acids, and organic waste, e.g., food waste or waste products from farming or forestry. Also, corn, sugar cane, vegetable oil, and some other types of biomass may be applied. One practical way to add bio-based PP as a fraction of the handle shell material is to apply the so-called bio-attributed concept, where the bio-based fraction of the PP is calculated from the mass balance approach (or model). This means that the fraction of bio-based PP in each handle shell may not be known, but the average fraction over some time, or for a certain number of handle shells, is well known. The supplier of the PP raw material to the molding, will comply with the relevant standards (such as the series of ISO 16620 standards: ISO 16620-1:2015, ISO 16620-2:2019, ISO 16620-3:2015, ISO 16620-4:2016, ISO 16620-5:2017) related to the mass balance model. Applying this approach means that in order to know the bio-based fraction of PP in a specific handle shell, or a specific batch of handle shells, an analysis, such as an analysis of the carbon-14 content, may be necessary. The rest of the PP may come from fossil resources. Some of the PP could also be based on recycled PP.

Some changes in the design of the endoscope handle have been found to simplify the manufacturing of the endoscope handle and have enabled a shift from previous used polymers for the handle shells, such as ABS, into the use of PP, including bio-based PP. Recycled PP may comprise a combination of bio-based PP and fossil-based PP. As more and more bio-based PP is applied, the fraction of bio-based PP in recycled PP will increase. The content of bio-based PP does not influence the use of additives which may be added to the PP to provide specific, e.g., mechanical, characteristics to the endoscope handle material.

The PP of the housing shells may comprise at least 20% bio-based PP, alternatively, at least 40% bio-based polypropylene. The fraction of bio-based PP may also be at least 60%, or at least 80%. A higher fraction of bio-based PP in the handle shell, means that the carbon dioxide emissions related to the endoscope handles during their life cycle, will be lower. The housing shells may consist essentially of PP. Processing additives may be added as is known in the art. Alternatively, the housing shells may comprise at least 60% PP, preferably more than 70% PP, of which at least 20% is bio-based PP. Fibers, such as natural fibers, may be added to modify physical properties, such as strength. In one variation, the housing shells may be overmolded to provide a desired texture feel or to provide color to portions of the handle. The overmolding material may or may not be PP.

In one variation of the present embodiment, the handle comprises at least one component and a component fastener/interface configured to secure the at least one component to a handle shell inside the compartment. To further reduce or eliminate use of adhesives, part of the component fastener is made in one-piece with, and of the same material as, a handle shell. The component fastener extends from the shell into the compartment. The at least one component is selected from the group comprising: a roller for bending the bending section by pulling a steering wire, a wire pipe fastener connecting the wire pipes to a handle shell, and/or an enclosure for a printed circuit b.

It has been found that this embodiment can provide a single-use endoscope having a lower carbon footprint. The use of PP for the handle shells has not previously been considered because gluing on PP is difficult. PP has a low surface energy—typically only 29 mN/m. To achieve “wetting” or adhesion, with most adhesives surface energy above 36 mN/m is needed. High surface energy plastics, such as ABS and polycarbonate, bond well with standard conventional adhesives and tapes possessing lower surface energy. But the use of PP for the housing shells will reduce the carbon footprint compared to the use of ABS. When part of the PP is bio-based, i.e., originates from renewable feedstocks instead of fossil feedstocks, the carbon dioxide footprint will be reduced further.

Additionally, a simpler manufacturing process is achieved by having fasteners and interfaces integral with and made of the same material as, a housing shell. This facilitates fast and simple assembly of components into the housing shells, as well as fast and simple assembly of two housing shells into a handle for an endoscope.

Further, the application of such integrated fasteners and interfaces makes it possible to reduce or even avoid the use of glue/adhesive materials in the assembly process. This enables simpler manufacturing and recycling processes of the different materials applied in an endoscope handle.

In another variation of the present embodiment, the insertion cord includes an insertion tube. The handle comprises the steering wire actuator and the wire pipe fastener. A first steering wire and a second steering wire are both connected to the steering wire actuator and run through the insertion cord so that manipulation of the steering wire actuator causes bending of the bending section. A first and a second wire pipe extend from the wire pipe fastener to a distal end of the insertion tube, the first and the second steering wire running inside the first and the second wire pipe, respectively.

The advantage of the wire pipe fastener is that the steering wires between the wire pipe fastener and the steering wire actuator can be arranged to propagate away from inner surfaces of the handle shells. This leaves more space for e.g., tools fixating steering wire ends to the steering wire, e.g., by crimping, during the manufacturing of the endoscope.

Further, this design of the wire pipe fastener ensures that the same wire pipe fastener can be applied in different types of endoscopes having different dimensions of the steering wire actuator.

In a further variation of the present embodiment, the first and the second channel each have a centerline extending in parallel with the first and second axis, respectively. This has the advantage of simplifying the positioning of the first and second wire pipes in a selected mutual angle, during manufacturing of the endoscope.

In a variation of the present embodiment, the handle comprises an encasement having a wall part to which the wire pipe fastener is attached. Combining the two parts has the advantage of reducing the number of different parts which needs to be handled and assembled during manufacturing, potentially reducing the use of adhesives. Also, as the encasement is typically larger than the wire pipe fastener, fixation of the wire pipe fastener to the encasement enables a strong and stable connection between the wire pipe fastener and the handle shells. Such a strong and stable connection is important for the bending performance when manipulating a bending lever. The wall part of the encasement may have a planar surface, on the part exterior to the encasement, to which the wire pipe fastener is attached.

In a variation of the present embodiment, the wall part of the encasement and the wire pipe fastener are made in one piece of material. This may be done by molding the two parts in one piece of material, whereby the wall part and the wire pipe fastener are fused together. This will reduce the costs by combining two molding processes into one. Further, combining the two parts may save space inside the handle, as separate means, such as glue or screws, for fixation of the parts to the handle shells, can be avoided.

In a variation of the present embodiment, a slot is placed at the position C where the first and the second steering wires are crossing each other. This slot has the advantage of forcing the steering wires to cross at the position C, also when another size of the roller is applied for a different endoscope model. Having the crossing at the same position between different endoscope models, may streamline the manufacturing process.

In a further variation of the present embodiment, a structure forming the slot projects from and is attached to the outer wall part of the encasement. When these two parts are attached to each other, the assembly process of the endoscope handle or actuator may be faster. The structure forming the slot may comprise two walls spaced apart by the slot.

In a variation of the present embodiment, the outer wall part of the encasement, the wire pipe fastener, and the structure forming the slot, are made in one piece of material, e.g., by fusing the parts together, e.g., in a molding process forming the parts in one single piece of material. This will reduce the number of parts, thereby making the manufacturing cheaper.

In a variation of the present embodiment, the channels are arranged in parallel displaced planes. Preferably, the bottoms of the channels are arranged in parallel but displaced planes. Both planes may be perpendicular to, or substantially perpendicular to, an axis of rotation of a roller being part of the steering wire actuator. One advantage of the displaced planes is that it ensures that the two steering wires will not get into contact between the wire pipe fastener and the steering wire actuator.

In a variation of the present embodiment, the wire pipes are extending proximally from the wire pipe fastener and passed the position C. An advantage of this is that if the steering wires should get close to each other in the position C, there is no possibility that they will slide against one another, and thereby reduce the bending performance of the endoscope.

In a variation of the present embodiment, the distance between the steering wire actuator and the position C is larger than the distance between the wire pipe fastener and the position C, preferably the distance between the steering wire actuator and the position C is at least the double of the distance between the wire pipe fastener and the position C. This is an advantage as the fixation of a steering wire end to the steering wire itself preferably takes place between the steering wire actuator and the position C. So, this will leave more space for any tool, E.g., crimping tool, to be applied for this process.

In a variation of the present embodiment, the channels each having at least one inner surface provided with ribs, the ribs are preferably extending in a direction being transverse to the direction of the first or second axis of the wire pipe placed in the respective channel. The ribs will improve the attachment of a glue inside the channel.

In a variation of the present embodiment, the wire pipe fastener is provided with a lid part covering at least part of the first channel and of the second channel. The lid part may be provided with holes for application of glue into the channels after placement of the lid part. The lid part may be made from a transparent polymer, facilitating curing of applied glue by UV light. The lid part may be applied for pressing the wire pipes into the correct position or for keeping the wire pipes in the correct position during the gluing process.

The aforementioned variations, and others described below, may be combined. For example, the handle may be manufactured from PP or another material, while incorporating part of the component fastener in a one-piece part. The wire pipe fastener may be as described above or may comprise a different design of a wire pipe fastener. The handle shells may be affixed to each other with the especially configured fasteners or, if made from ABS, with adhesives. The handle shells may at least partially be fused to each other, with or without adhesives, using fusing processes such as ultrasonic bonding or heat-steaking. The housing shells may be integrally formed with the wall part of the encasement and the wire pipe fastener.

In a second aspect, the disclosure relates to a system comprising an endoscope according to the first aspect and variations thereof, a monitor and a control unit.

In a third aspect, the disclosure relates to a method for assembling an endoscope. In one embodiment according to the third aspect, the method comprises: providing a polypropylene material comprising at least 20% bio-based polypropylene; injection molding two housing shells from the polypropylene material, where the housing shells are molded with integrated fasteners configured for connecting the two housing shells to each other to form a handle of the endoscope, and, the housing shells are molded with at least one integrated interface configured to connect a housing shell to at least one component selected from a group comprising: a roller for bending the bending section by pulling a steering wire, and a wire pipe fastener connecting the wire pipes to a handle shell; connecting the at least one component to the at least one interface; and assembling the two handle shells with the fasteners to form an internal compartment of the handle with the component connected to a housing shell and positioned in the internal compartment.

### BRIEF DESCRIPTION OF THE FIGURES

The above-mentioned embodiments and variations, features and advantages thereof will be further elucidated by the following illustrative and nonlimiting detailed description of embodiments disclosed herein with reference to the appended drawings, wherein:

FIG. 1 is a schematic view of a visualization system including an endoscope and a monitor with a control unit;

FIG. 2A is a side view of a variation of a handle;

FIG. 2B is a back view of the handle of FIG. 2A;

FIG. 3 is an exploded view of a handle housing of the handle of FIGS. 2A and 2B;

FIG. 3A is an enlarged view of a roller interface first shown in FIG. 3;

FIG. 4 is a perspective view of a handle shell of FIG. 3;

FIGS. 5A-5D are detailed views of a snap-fit for assembling the handle of FIG. 3;

FIGS. 6A-6C are detailed views of shell component fasteners;

FIG. 7 is a perspective view of a handle shell of the handle of FIGS. 2A and 2B provided with components of an endoscope;

FIG. 8 is a perspective view of the steering wire actuator;

FIG. 9 is a perspective view of a suction valve;

FIG. 10 is a perspective view of the handle shell of FIG. 7 provided with further components of the endoscope;

FIGS. 10A to 10D illustrate additional components arranged in an inner compartment of the handle of the endoscope;

FIG. 11 is a perspective detailed view of the handle shell of FIG. 10 showing a steering wire actuator and a wire pipe fastener;

FIGS. 12 to 15 are a perspective detailed views of an encasement supporting the wire pipe fastener of FIG. 11;

FIG. 16 is another perspective view of an encasement supporting the wire pipe fastener of FIG. 11;

FIGS. 17 to 19 are perspective detailed views of a variation of the embodiment of the encasement of FIG. 11;

FIGS. 20 to 22 are perspective views of a embodiment shown in FIG. 11, in which the wire pipe fastener is arranged on a support platform;

FIG. 23 is a perspective view of a bending section; and

FIG. 24 is a flowchart.

In the drawings, corresponding reference characters indicate corresponding parts, functions, and features throughout the several views. The drawings are not necessarily to scale and certain features may be exaggerated in order to better illustrate and explain the disclosed embodiments. For simplicity, this disclosure will focus on a two-way bending endoscope, but the disclosure is relevant for, and covers, also a four-way bending endoscope.

### DETAILED DESCRIPTION

The term “distal” is defined to be in the direction toward the patient, and “proximal” is defined to be in the direction away from the patient. For the handle of the endoscope, the distal end will be the end where the insertion tube is connected, and the proximal end is the opposite end. Further, the expression “handle” may be a positioning component fastener, or component fastener, which functions to control the position of the insertion cord. The handle, or positioning component fastener, may be an component fastener operated by a robotic arm, or it may be a handle operated by the hand of an endoscope user.

A steering wire refers to a wire extending from the steering wire actuator to the distal end of the bending section. If the same steering wire is bent at the distal end and returns to the steering wire actuator, the returning portion is referred to as a second steering wire. In other words, a steering wire is a portion of wire extending once through the endoscope, so that if two portions extend through the endoscope they are referred to as two steering wires even if they are connected.

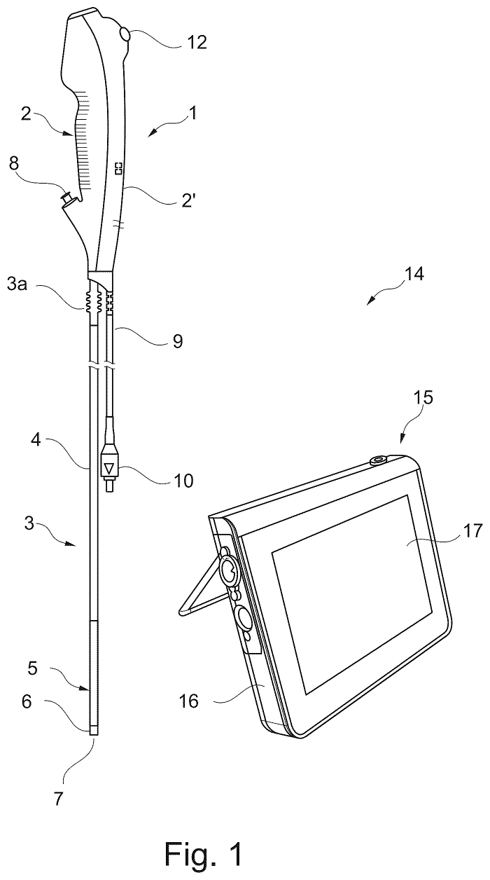

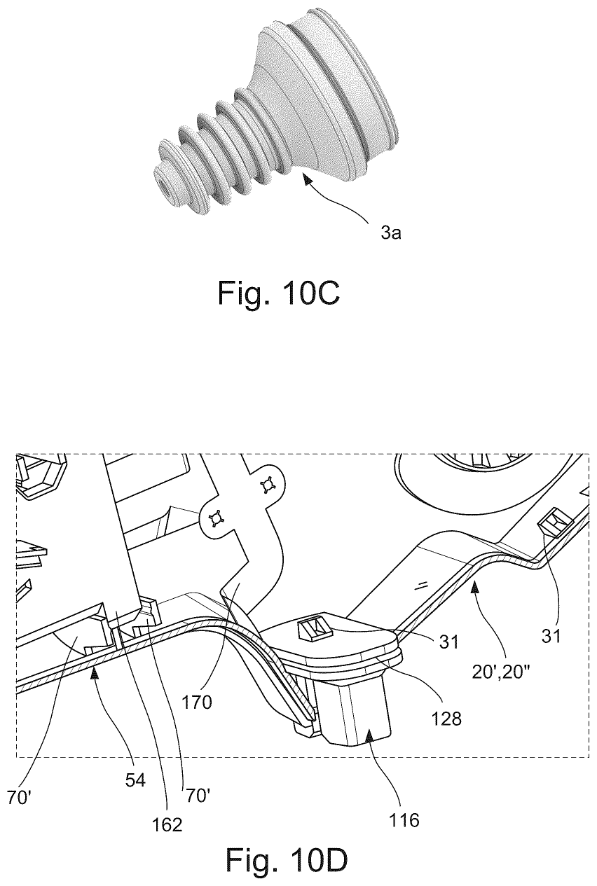

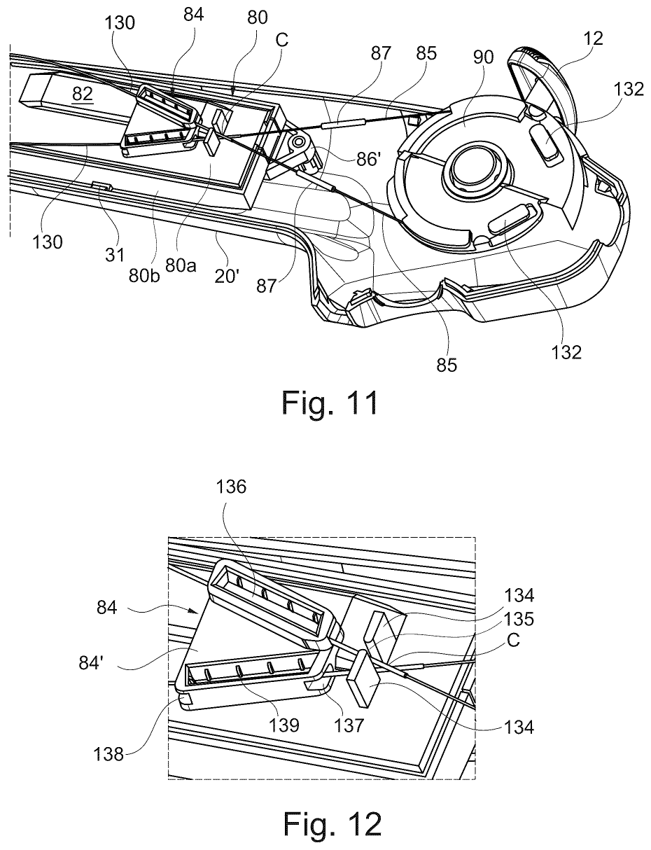

FIG. 1 illustrates a visualization system 14 comprising an endoscope 1 and a monitor 15. The endoscope 1 comprises a handle 2, an insertion cord 3 and an electrical cable 9 with a connector 10 for connecting the endoscope 1 to the monitor 15. The insertion cord 3 is the part to be inserted into a body lumen during an endoscopic procedure. The insertion cord comprises, extending distally from the handle, in order, a main tube 4, a bending section 5 and a distal tip 6. The handle 2 may comprise a housing 2′ with an entrance to a working channel 8 running through the insertion cord to the distal tip. The handle also comprises a bending lever 12, which can be used for bending the bending section 5. The distal tip 6 comprises a camera 7 including an image sensor and light emitters in the form of one or more light emitting diodes (LEDs) or the end of one or more optical light fibers. A resilient insertion cord relief 3a is bonded to an insertion cord bushing, shown in FIG. 10B, to reduce the risk of the insertion cord kinking in the proximal end. A perspective view of the insertion cord relief 3a is shown in FIG. 10C.

The monitor 15 may be combined with an electronic circuit for receiving and processing the image stream from the camera as well as a processor for image processing, user interface, storage of images etc. But the monitor, the electronic circuit and the processor may also be separate parts. The electronic circuit and the processor are also referred to as a control unit 16. The monitor 15 as shown includes a display screen 17. The display screen 17 may also be a physically separate component communicatively connected to the monitor 15.

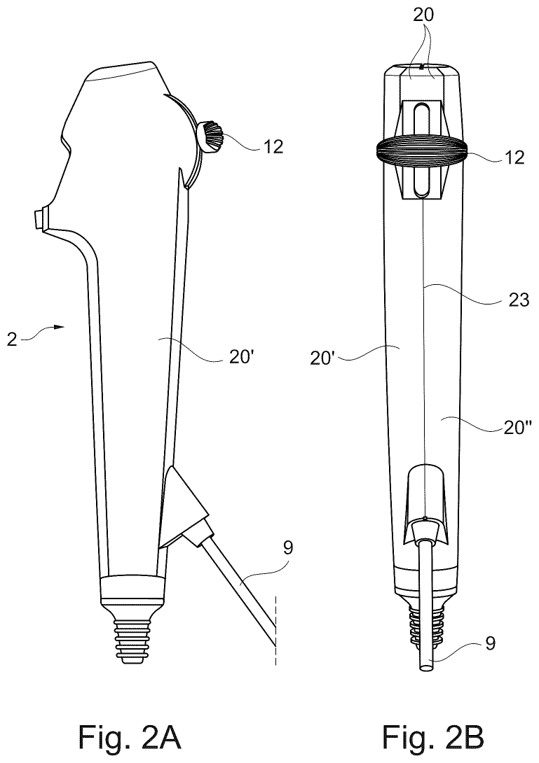

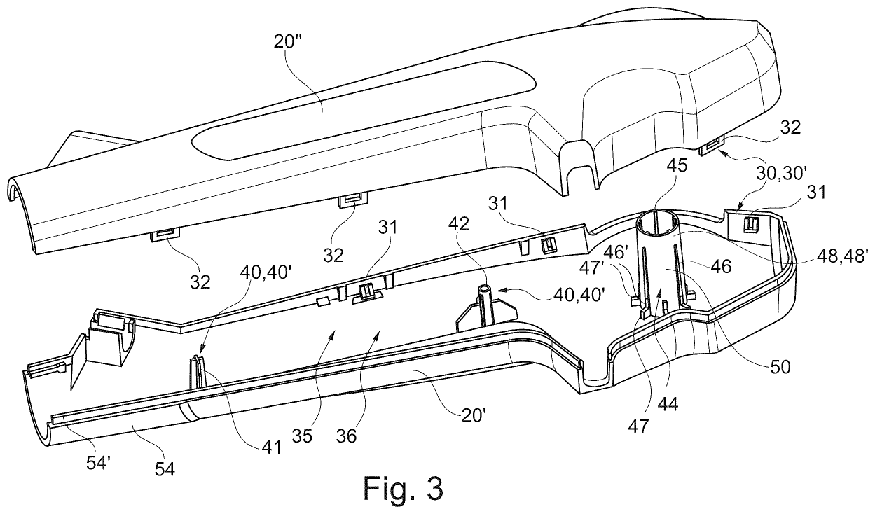

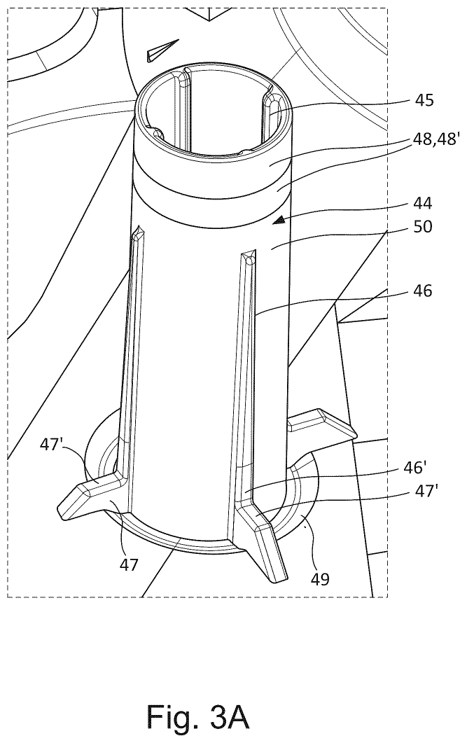

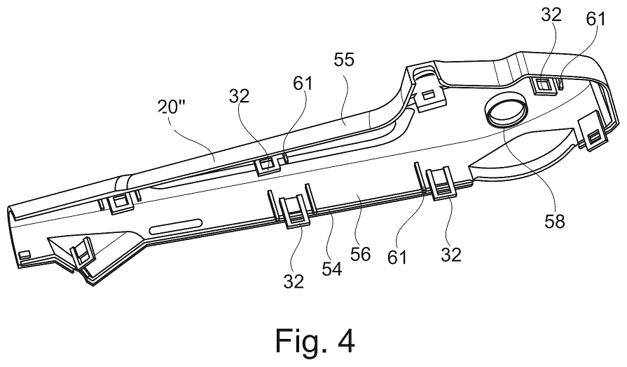

FIGS. 2A and 2B are side and back views of a variation of the handle housing 2′. The housing 2′ is made from two housing shells 20′, 20″. The housing shells 20′,20″ each have an outer surface. The shapes of the outer surfaces may be symmetrical in relation to a plane in which they are connected when assembled. In that case, the periphery of the handle in that plane may follow a handle split line 23 in which the two shells are connected. FIG. 2A shows primarily the left housing shell 20′, seen from a user of the endoscope holding the handle 2 in the left hand with the left-hand thumb on the bending lever 12. When held in this way, FIG. 2B shows the endoscope seen from the back, with the left housing shell 20′ to the users left side and the right housing shell 20″ to the users' right side. The assembled housing shells 20′, 20″ form an inner compartment 36 (FIG. 3). Inner surfaces of the housing shells 20′, 20″ face this compartment. The inner surfaces of the housing shells extend opposite to the outer surfaces of the housing shells.

FIG. 3 is a perspective exploded view of the housing 2′. The housing shells 20′, 20″ may be symmetrically shaped on their outer surfaces, but the inner surfaces of the two housing shells may differ as these can be molded with a number of shell fasteners 30 for assembly and interconnection of the housing shells 20′, 20″ to each other to form the housing 2′ and component fasteners 40 for the connection of different components of the endoscope to the housing shells. A shell wall 54 is shown. A thin portion, or lip 54′, of the shell wall 54 extends past the split line 23 coextensively with the inner surface of the housing shell 20′. The lip 54′ is thin, potentially thinner than ½ the thickness of the shell wall 54. A corresponding lip of the housing shell 20″ may extend coextensively with the outer surface of the housing shell 20″ so that the lips overlap when the housing is assembled. Due to the small thickness, alignment means and the fasteners may be positioned to form the split line 23. Both shell fasteners 30 and component fasteners 40 may be integrated with the housing shells 20′, 20″. The shell fasteners 30 and component fasteners 40 mechanically attach parts and components to each other without adhesives. While their inclusion in the handle shells increases molding complexity, their inclusion addressess problems that arise when a flexible material, such as PP, is used to mold the handles. They also contribute to the reduction in the carbon footprint by allowing reductions in the use of adhesives. Because the walls of the handle shells are more flexible than when molded from ABS, the edges of the walls might not always align perfectly, and this creates a user perception of lesser quality. The shell fasteners 30 and component fasteners 40 create a synergistic three-dimensional network 35 of structural supports that provide rigidity and attachment features for the handle shells and the handle components.

Both shell fasteners 30 and component fasteners 40 may comprise snap-fits, press-fits, heat staking, or any other fastening method where a major part, or the entirety, of the shell fastener 30 or the component fastener 40 may be molded as an integrated part of a housing shell 20′, 20″. This could also involve ultrasonic welding, i.e., the shell fasteners or component fasteners could be designed attachment by ultrasonic welding.

Shell fasteners 30 in the form of snap-fit shell fasteners 30′ are illustrated in FIG. 3. The members of the snap-fit shell fasteners are arranged on the inner surfaces of the housing shells close to the handle's split line 23 so that they maintain the edges forming the split line 23 in good alignment in spite of the flexibility of the walls of the housing shells. The wall thickness of the housing shells may be less than 3 mm, preferably between 1.5 and 2.5 mm, and even more preferably between 1.5 and 2.2 mm, to provide sufficient support for the shell fasteners 30 and component fasteners 40 while minimizing the amount of plastic material.

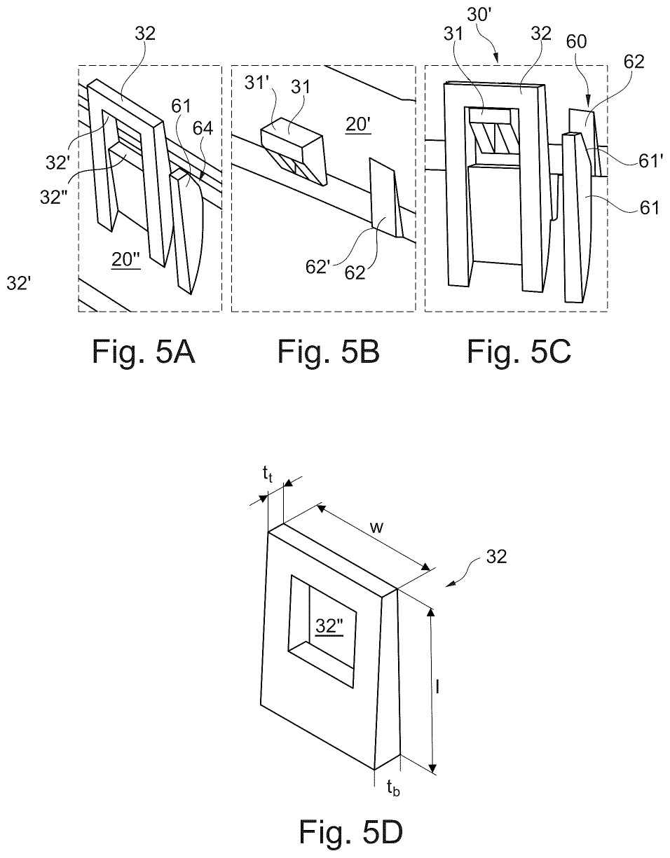

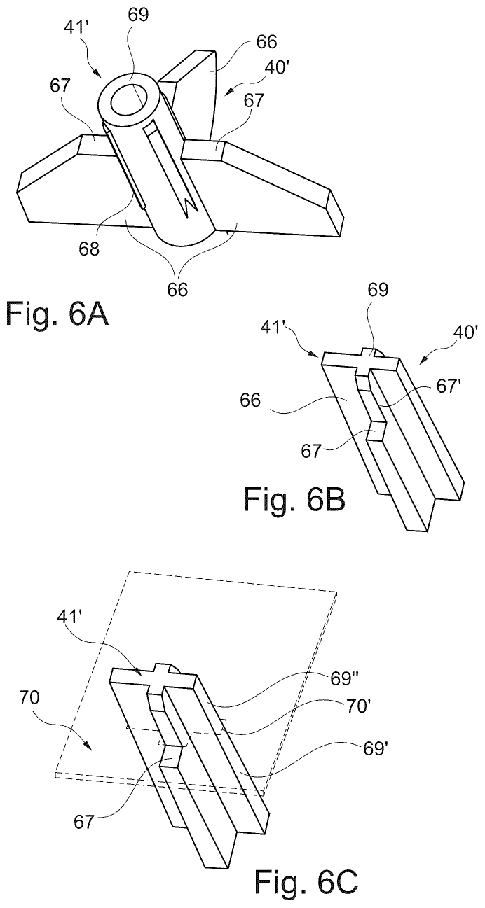

The snap-fit shell fasteners 30′ comprise a snap lug 31 and a snap hook 32. The snap lug 31 is placed on one housing shell and a corresponding snap hook 32 is placed on the other housing shell. When the housing shells 20′, 20″ are pressed together the snap hooks engage with the snap lugs to attach the housing shells to each other. In the range of 6-12 snap-fit shell fasteners 30 provide a strong and stable connection between the housing shells. In one variation, 7-9 snap-fit shell fasteners 30′ connect the two housing shells. The snap-fit shell fasteners 30′ can be described as protrusions extending inwardly from the inner surfaces of the housing shells, the snap hook comprising a recess 32″ into which the snap lug 31 penetrates. The snap lug has a retention surface 31′ that abuts a corresponding retention surface 32′ of the snap hook (shown in FIGS. 5A and 5B), the retention surfaces preventing separation of the housing shells. The snap hook extends from the wall of the housing shell and traverses the split line 23. Due to the extension and the use of PP material, the snap hook is resiliently flexible so that it can flex inwardly when engaged by the snap lug during assembly and when its retention surface translates past the retention surface of the snap lug it flexes in the opposite direction so that its retention surface abuts the retention surface of the snap lug. Preferably, an outwardly facing surface of the snap hook lies substantially on a common plane with the inner surface of the housing shell. Although as shown all the snap lugs 31 are on one housing shell and all the snap hooks 32 are on the other housing shell, this is not necessary and the snal lugs and hooks can be positioned in both housing shells. The snap-fit shell fasteners 30′ are described in more detail with reference to FIGS. 4 and 5A-5D.

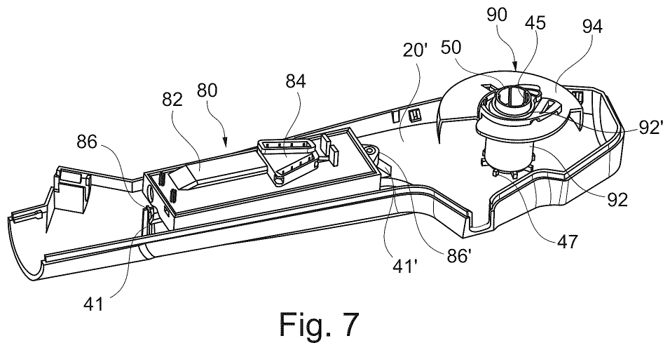

Component fasteners 40 in the form of press-fit component fasteners 40′ are also illustrated in FIG. 3. A press-fit component fastener may comprise a stud or rod 41, 41′. The press-fit component fasteners 40′ are described in more detail with reference to FIGS. 6A-6C. Component fasteners 40 may be referred to as interfaces configured to secure components to at least one of the housing shells. The interfaces may be integral with and comprising the same material as one or the other of the housing shells, therefore the integral interfaces are made in one-piece with one or the other housing shells.

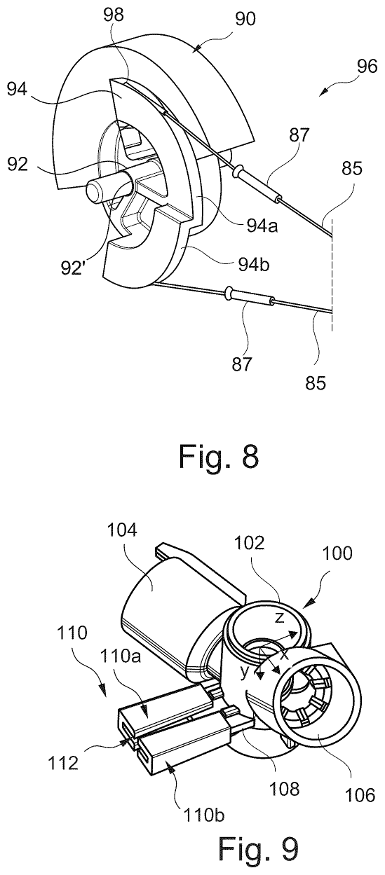

Another component fastener shown in FIG. 3 comprises a roller component fastener 44, which provides an axle for a roller and is molded with a housing shell 20′, 20″. An enlarged view of the roller component fastener 44 is shown in FIG. 3A. The roller is rotatably mounted onto the roller component fastener 44. The roller component fastener 44 comprises inner ribs 45, outer ribs 46, and support walls 47 extending outwardly from the outer ribs 46 along the inner surface of the housing shell. The outer ribs 46 and the support walls 47 provide rotation surfaces 46′,47′ which, together with a rotation surface 48′ at a free end 48 of the roller component fastener 44 allow the roller to rotate in a stable manner with minimal friction. Friction is undesirable because it can cause the user to fatigue while navigating the endoscope in the patient. The ribs 45, 46 provide rigidity to a cylindrical core 50 of the roller component fastener 44 without requiring that the wall thickness of the cylindrical core 50 be increased to compensate for the use of PP material instead of ABS material. Portions of the cylindrical core 50 and the outer ribs 46 intermediate the rotation surfaces 46′ and 48′ do not contact the roller. The rotation surfaces 47′ limit the axial translation of the roller. The cylindrical core 50 comprises a tubular wall. The support walls 47 are relatively small. In FIG. 6A the core is smaller and the support walls are larger. The support walls 47 can extend outwardly from the core instead of the outer ribs 46. However, from a molding perspective, it is preferred that the support walls 47 extend outwardly from the outer ribs 46 as it simplifies mold construction. The ribs may be placed symmetrically around a plane going through the center axis of the roller and through a center of a wire pipe wire fastener 84 (see FIG. 7). The plane corresponds to a steering wire tension direction. Thereby, the pull force from the steering wires on the roller will be equally distributed on two ribs (when the roller has four ribs with 90 degrees between them). The size an number of ribs can be increased or decreased, in part depending on the type and size of endoscope being assembled. A groove 49 surrounding the cylindrical core 50 into the housing shell material, where the roller is connected, is provided for molding purposes, to avoid that the material thickness be too large at the circular joint, which could cause the creation of sink marks during molding. Sink marks could be visible on the external surface of the handle and are therefore undesirable. The support walls 47 extend past the groove 49 to support the cylindrical core 50.

FIG. 4 is a perspective view of one of the housing shells. In addition to the snap hooks 32, the housing shell illustrates the shell wall 54 having an outer surface 55 opposite an inner surface 56, a circular wall 58, and first alignment parts 61. The circular wall 58 comprises a cavity therein receiving, and providing rotational support for, an axle of the roller. The first alignment parts 61 are described in more detail with reference to FIGS. 5A-5D.

Referring now to FIGS. 5A-5D, FIG. 5A illustrates the snap hook 32 of the snap-fit shell fasteners 30′ and alignment means 60 positioned along the handle split line 23. The alignment means 60 may be placed close to the snap-fit parts but do not need to be. The alignment means ensure that the outer surface 55 of the assembled handle is as smooth as possible without any significant edge in the transition from one housing shell to the other. In one variation, the alignment means comprises the first alignment part 61 and a second alignment part 62. The first alignment part 61 is a protrusion extending from the inner surface 56 of the wall 54 of the housing shell. The first alignment part 61 and the housing shell may form a receptacle 64, shown in FIG. 5A, with dimensions configured to receive the second alignment part 62, shown in FIG. 5B, which is integrated with the other housing shell. The receptacle 64 may be U-shaped. The retention surfaces 31′ and 32′ are also shown.

The first alignment part 61 may comprise a slanted surface 61′ and the second alignment part 62 may comprise a slide edge 62′ configured to, potentially, slide onto the slanted surface 61′ so that the slanted surface 61′ can push onto the slide edge 62′ and thus align the edges of the housing shells. The receptacle 64 may be omitted.

The number of alignment means 60, i.e., pairs of a first alignment part 61 and a second alignment part 62, may be in the range of 5-13, preferably in the range of 6-10. The combination of the snap-fit shell fasteners 30′ and the alignment means 60 form the three-dimensional network 35 of structural supports that has the effect that when the two housing shells are snapped together, they cannot be displaced relative to each other in any direction. Also, along the handle split line 23 the connection will be firm and robust. By preventing displacement, it is also prevented that the shells will un-snap and separate. The three-dimensional network comprises a plurality of alignment means and snap-fit shell fasteners 30′.

FIG. 5D shows some example dimensions for the snap hook 32. The snap hook 32 may have a length l from the connection to the housing shell 20′, 20″ to the top in the range 6-12 mm, e.g. about 9 mm, preferably 9+/−1 mm. The width w may be in the range 6-9 mm, e.g. about 7 mm, preferably 7+/−1 mm. The thickness at the bottom tb, where the connection to the housing shell is, may be in the range 1-3 mm, e.g. 1.6 mm, preferably 1.6+/−0.2 mm. The thickness at the top tt is in the range 0.6-2 mm, e.g. 1 mm, preferably 1.0+/−0.2 mm. The snap hook 32 may comprise a resilient protrusion which after being bent returns to its prior position. The resilient protrusion, or tab, comprises a recess (a through-hole is shown although the recess may be an indentation). During assembly the snap lug bends the tab until the snap lug at least partially penetrates the recess, allowing the tab to return to its prior position. These dimensions have been found to provide a good balance to achieve both the flexibility for connecting with the snap hook 32 in a simple and easy way and the strength to maintain a strong and robust connection between the two housing shells 20′, 20″. For comparison, the average wall thickness of a housing shell 20′, 20″ may be in the range 1-4 mm, preferably in the range of 1.5-2.5 mm, and even more preferably in the range of 2.0+/−0.2 mm.

Referring now to FIGS. 6A-6C, these figures illustrate in detail elements of the press-fit component fasteners 40′ including the press-fit stud or rod 41, 41′ for connecting components to a housing shell 20′, 20″. The press-fit studs 41, 41′ are molded in one-piece with, and extend from, a respective housing shell 20′, 20″. Attached components comprise a corresponding stud receiver 70′ of a component 70, schematically shown in FIG. 6C. The press-fit studs 41, 41′ are provided with support walls 66, for increased stability and stiffness of the connection and to spread the load over a larger area of the wall of the housing shell, and with support surfaces 67 for controlling correct positioning of the respective component in the compartment 36 and in relation to the housing 20. The press-fit rods may also be provided with component fastener ribs 68 extending from a core 69. This will improve the strength of the press-fit connection.

As shown in FIG. 6A, the core 69 comprises a cylinder and the component fastener ribs extend longitudinally thereon. Four component fastener ribs 68 are shown and they are shown equally distributed around the core 69. However, more or fewer component fastener ribs 68 may be provided and they can be distributed unevenly around the core 69. An upper surface of at least one wall defines the support surface 67 of the the press-fit stud 41′. A press-fit stud 41 may be used with a press-fit stud 41′ to hold a component. Due to the cylindrical core, the press-fit stud 41′ may allow rotation of the component, but the rotation is prevented by the press-fit stud 41. Together, the press-fit studs 41, 41′ facilitate securement of the component while the press-fit stud 41′ may allow for some variance in the positions of the stud receivers in the component.

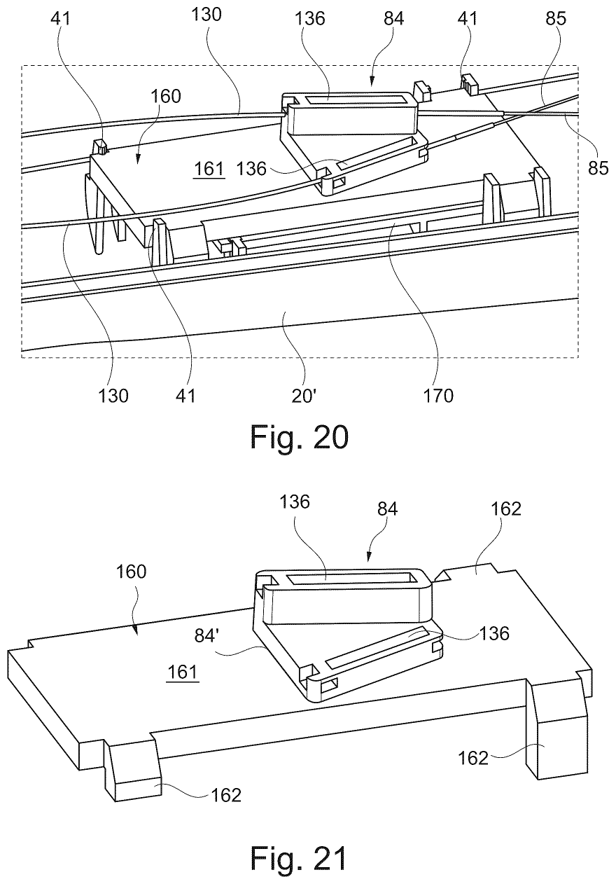

In FIG. 6B, the core 69 of the press-fit stud 41 comprises an intersection of the walls 66 and the ribs are omitted. One or more of the support walls 66 may comprise a cut-out 67′ that defines the support surface(s) 67. As shown in FIG. 6C, the press-fit stud 41 (and also the press-fit stud 41′) comprises a base portion 69′, extending between the housing shell and the support surface 67, and an upper portion 69″, extending from the support surface 67 away from the housing shell. A stud receiver 70′ of a component 70 slides through the upper portion until the component cannot slide further due to the support surface. A washer may be interposed between the component and the support surface 67. The press-fit stud 41 can be used to connect a printed circuit board (PCB), e.g. the component 70 in FIG. 6C, and/or a support platform, e.g. the support platform 160 in FIG. 20, which support platform may hold the printed circuit board and/or a wire pipe fastener 84 (see FIG. 7). The support platform can be a major surface of have a box or an encasement 80 for providing a watertight sealing for the PCB. The wire pipe fastener 84 may be placed on an outer surface of the support platform. There can be several different parts of the fasteners connecting the support platform to the handle shells. This may include press-fits between the support platform and a handle shell. It may also include snap-fits in relation to one or both housing shells 20′, 20″. A plurality of interfaces fasteners may provide alternative or additional alignment means since they may prevent relative movement between the housing shells, particularly when positioned near the edges of the housing shells.

FIG. 7 illustrates an example of components mounted on a housing shell. The components comprise an encasement 80 and a roller 90. The encasement 80 is provided with press-fit receivers 86 and 86′ which are configured to receive, respectively, the press-fit stud 41 and the press-fit stud 41′. The roller 90 comprises a cylinder 92 comprising a receiver 92′ that slides over the cylindrical core 50 until it reaches the rotation surfaces 47′ of the support walls 47. The cylinder 92 functions as an axle for a steering wire support wall 94 of the roller 90. The encasement 80 comprises a protruding enclosure portion 82 and a wire pipe fastener 84. Alternatively, the encasement 80 may be secured to the housing with fasteners, such as the snap-fit fasteners, or combinations of snap-fit fasteners and press-fit fasteners.

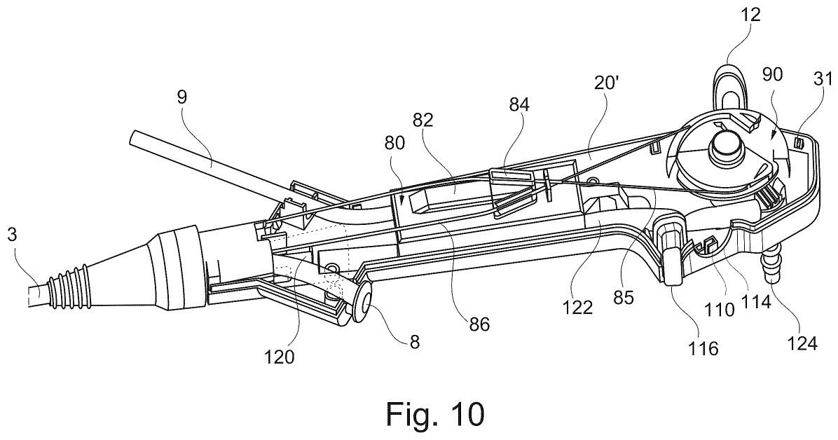

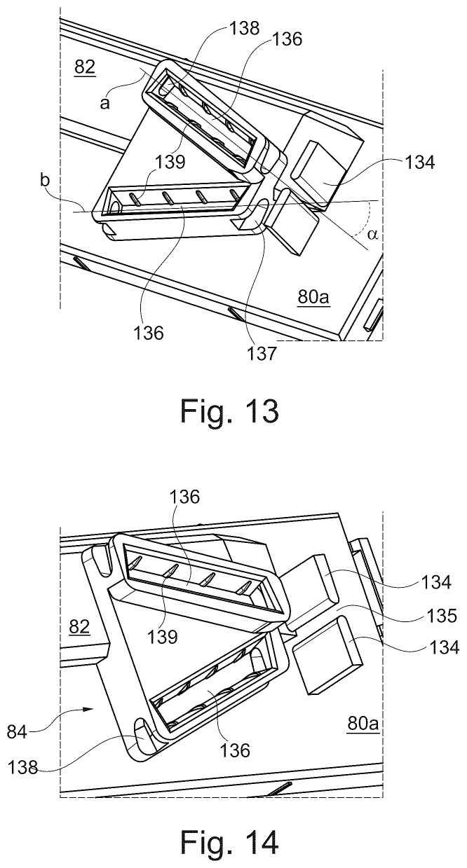

A perspective view of a variation of the steering wire actuator 96 including the roller 90 is shown in FIG. 8. The roller 90 is connected to the steering wires 85. Each of the two steering wire ends are connected around fixing structures in the roller 90, and each steering wire end is then guided back and secured to itself by crimps 87. The steering wire support wall 94 comprises two planar portions 94a, 94b offset from each other, each comprising a wire drum curved surface 98 upon which a steering wire 85 curves. The steering wires 85 are moved by rotation of wire drum curved surfaces 98, which are a circular arc surfaces or a curved surface placed on the roller 90 and supporting the steering wires. The fixing structure may be a hook (see FIG. 11) or a small hole (not shown) through which the steering wire is threaded. The steering wire actuator 96 works for a two-way bending endoscope. For a four-way bending endoscope two independently rotatable wire drums may be used.

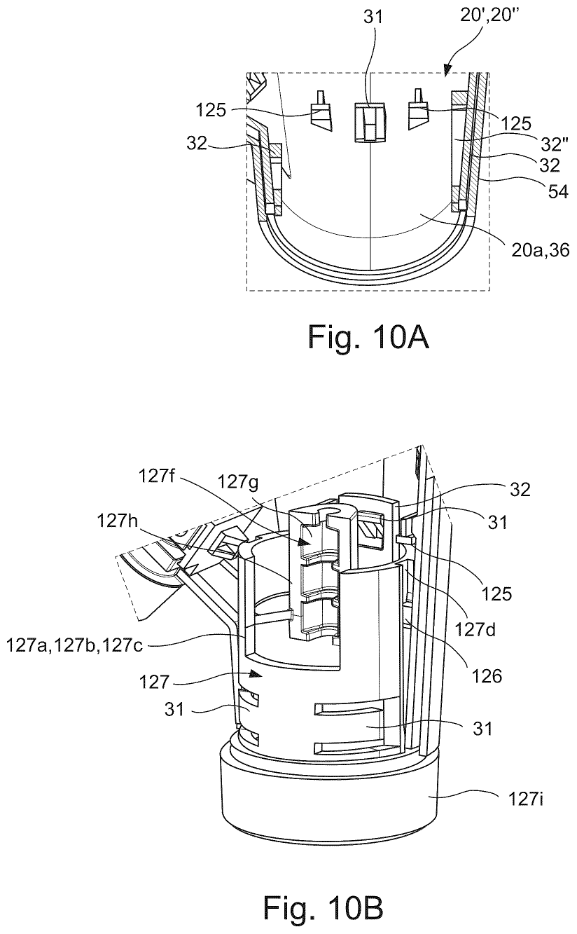

Another handle component is a suction valve 100, illustrated in part in FIG. 9. The suction valve 100 is mounted in a suction valve recess 114 (shown in FIG. 10) formed between the first housing shell and the second housing shell and comprises a valve body 102, a stem translatable in the valve body (not shown), an inlet 104, an outlet 106, and a fixation portion 108 extending orthogonally from the body. The stem is movable axially in the valve body to open and close the suction valve to control a suction through the suction valve and the working channel. A inner fixing portion 110 extends, starting from the housing shell wall, perpendicularly into the interior space. The fixing portion 110 comprises two U-shaped brackets 110a, 110b facing each other and forming a substantially rectangular or slot-shaped receptacle 112. The fixation portion 108 slides into the fixing portion 110 to secure the suction valve 100, potentially and preferably without adhesives. The suction valve, the fixation portions, and other details of structure to secure the suction valve to the housing are described in more detail in commonly-owned U.S. patent applications Ser. Nos. 18/824,143 and 18/824,157, titled “ENDOSCOPE COMPRISING A SUCTION VALVE,” the disclosures of said applications are incorporated herein by reference in their entirety.

Referring to FIG. 10, the components to be arranged in the compartment 36 may be one or more of the encasement 80, the roller 90 connected to the lever 12 and configured for bending the bending section 5 by pulling steering wires 85, the wire pipe fastener 84 connecting wire pipes 86 to the housing shell 20′, media buttons 116 e.g., actuatable to send a signal configured to cause the VPA to store video or an image, the suction valve 100 (the recess for the valve is shown) controlling suction through a working channel formed in part by a working channel tube 120, tubing 122 for suction, e.g., a tubing connecting the suction valve 100 to the working channel tube 120, and the working channel port 8 (or an Y-connector or biopsy connector) enabling insertion of tools into and through the working channel tube 120 of the endoscope. The suction valve 100 is also connected to a suction connector 124, which in use may be connected to a vacuum source such as a vacuum pump. The component fasteners 40 for connecting one or more components to a housing shell are an integrated part of at least one housing shell and may also comprise a corresponding part on the component. The two parts are in that case configured to connect with each other.

FIGS. 10A and 10B illustrate another component arranged in the inner compartment 36, in particular at the distal end of the housing 20. FIG. 10 shows the distal end 20a of the housing shells 20′, 20″ and in the distal end 20a the inner compartment 36 is configured to accommodate an insertion cord bushing 127, shown in FIG. 10B. The insertion cord bushing 127 is color coded to represent a characteristic of the endoscope, such as a technology type and/or an endoscope size. The handle 20 may be white and the bushing may be, for example, green, blue or red. The housing shells and the insertion cord bushing include snap-fit shell fasteners, illustratively including snap lugs 31 in the housing shells and snap hooks 32 in the insertion cord bushing 127. When the insertion cord bushing is placed between the housing shells, the recesses 32″ of the snap hooks 32 are penetrated by the snap lugs 31 to prevent removal of the insertion cord bushing 127. A pair of bushing stops 125 limit proximal movement of the insertion cord bushing 127 when they contact a proximal portion of the the insertion cord bushing 127. The housing shells and the insertion cord bushing may also include additional snap-fit shell fasteners, illustratively including snap lugs 31 in the the insertion cord bushing 127 and and snap hooks 32 in the housing shells 20′, 20″. The snap hooks 32 are supported by arms extending distally.

In the present embodiment, the insertion cord bushing 127 comprises a wall 127a that has a cut-out 127b defining a hole 127c in the wall 127a. The wall 127a also has a pair of longitudinal slots 127d in which a protrusion 126 of the housing shells 20′, 20″ may penetrate to prevent rotation of the insertion cord bushing 127. The insertion cord bushing 127 also includes, within a cavity defined by the wall 127a, a basin 127f into which a proximal end of the insertion cord 3 is adhesively bonded. A hole 127g is connected to the basin to allow cables from the insertion cord 3 to extend from the basin to the PCB 170 (see FIG. 20). The basin is molded in one piece with the insertion cord bushing. The same colored material is applied for the whole part. Therefore, the basin needs to have an open surface so that ultraviolet (UV) light can be applied to harden the adhesive. The basin, as shown, is formed in a cylindrical portion 127h of the insertion cord bushing 127. Optional ribs inside the basin 127f can provide an anchoring effect for the solidified adhesive. A distal end of the insertion cord bushing 127 is visible after assembly. The basin may be filled with ultraviolet (UV) hardenable adhesive which is hardened by illuminating the UV light through the hole 127c formed by the cut-out 127b, after the insertion cord has been arranged through the basin. In other embodiments, the adhesive may be other than IV hardenable adhesive, e.g. epoxy or other adhesive that cures with time. The insertion cord relief 3a, shown in FIG. 10C, is adhesively bonded inside the distal end of the insertion cord bushing 127. The insertion cord relief 3a may be of a color matching the insertion cord color.

FIG. 10D illustrates another component arranged in the inner compartment 36. In the present embodiment, one of the handle shells is provided with a wall 128 to supporting the media button 116. On the side of the wall 128 opposite to the media button 116, a snap lug 31 is arranged. On the other housing shell, a corresponding snap hook 32 is provided. When the housing shells are assembled, the recess 32′ of the snap hook 32 is penetrated by the snap lug 31 to secure the media button 116 in place. A portion of the PCB extends between the wall 128 and the wall 54 to receive a signal when the media button 116 is pushed. The signal is transmitted by the PCB to the VPA, which takes an action corresponding to the signal, such as recording an image or a video clip, for example having a duration corresponding to a time during which the media button is pressed. The PCB, or more generally, the flexible circuit board, is connected to a handle shell by press-fit component fasteners, e.g. a stud or rod 41, 41′, integrally molded with the handle shell. Also shown are press-fit component fasteners 70′, 162 to secure a support platform described with reference to FIG. 20.

FIG. 11 shows the handle shell 20′ with the roller 90 including the bending lever 12, attached, and fixing structures 132, for example steering wire hooks. Also shown is the encasement 80 for e.g., electronics. The encasement 80 is connected to the handle shell 20′ and may have the form of a box. The encasement 80 may be watertight e.g., to protect enclosed electronics. The encasement 80 may have a wall part, or support platform, 80a to which the wire pipe fastener 84 is attached. The steering wires 85 cross at point C. The protruding enclosure portion 82 is also shown.

The encasement 80 may be attached to the handle shell with press-fits or snap-fits. This facilitates a simple assembly process of the endoscope. The encasement may be assembled from two parts and may comprise apertures for e.g., electrical wire connections. The encasement may be a box that is a separate part and comprises a two major walls, e.g. a bottom wall and a support platform 80a, and circumferential wall(s) 80b between the support platform 80a and the bottom wall. Thus, the support platform 80a or the bottom wall may be a separate part that is attached to form the inner space. The support platform 80a may be press-fit or adhesively bonded to the circumferential walls 80b to enclose an inner space where an electronic circuit board may be arranged. The inner space may be sealed or unsealed. Unsealed inner spaces may be suitable for endoscopes with limited access to fluids, such as ENT endoscopes.

Alternatively, some walls of the encasement are part of the housing shell, e.g. the bottom wall. In an example, the encasement 80 has one part comprising one major surface also comprising the wire pipe fastener 84, e.g. the support platform 80a, and the circumferential wall 80b. An opposite major surface is formed by the handle shell 20′. In this way the encasement 80 may be formed from two molded parts, where one part is also a housing shell, and the other part also comprises the wire pipe fastener 84. Such construction reduces the number of attachments and the number of parts, reducing assembly costs. Attachment of the single part comprising the support platform 80a, the circumferential wall 80b, and the wire pipe fastener 84 to the housing shell encloses the inner space. The press-fit and/or snap fit parts can extend from the circumferential wall 80b to secure such a single part to the housing shell.

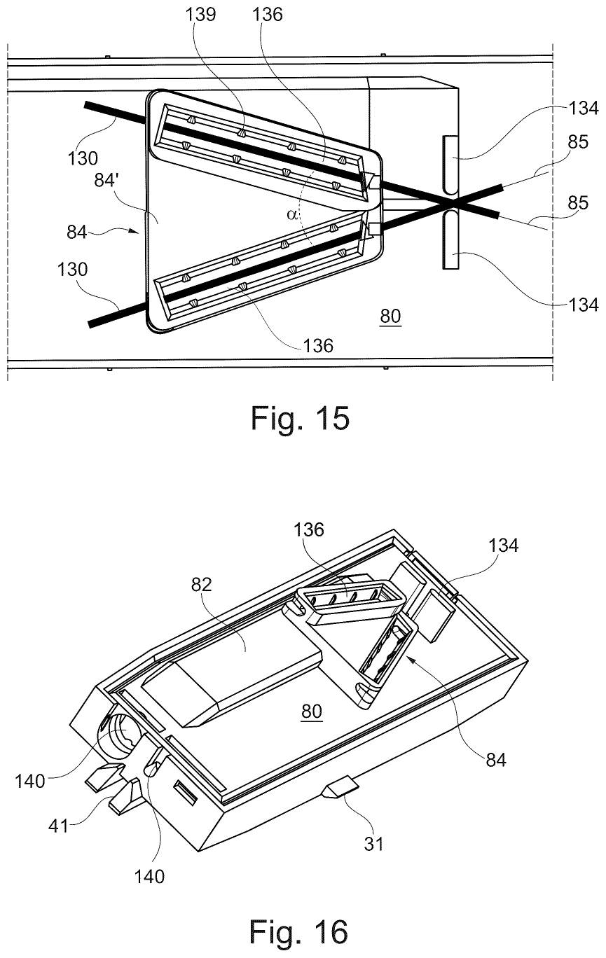

An example of the wire pipe fastener 84 is shown in FIGS. 12 to 16, where FIG. 12 is an enlarged view from FIG. 11. Two projections 134, in the form of walls, plates or rods, are placed distal to the wire pipe fastener 84 and form a slot 135 between them close to the point C where the steering wires cross. The projections will preferably have a curved surface facing the slot. The distance between the proximal end of the wire pipe fastener and the distal side of the projections 135 may be between 2-8 mm, preferably between 3-6 mm. In alternative examples, the projections 134 are omitted.

The purpose with the slot 135 is to limit the position of the point C where the steering wires are crossing. Such a fixed or substantially fixed position of the point C to a specific distance from the proximal end of the wire pipe fastener in the direction of the roller 90 facilitates that the same design of the wire pipe fastener 84 may be used with different diameters of the roller. This means that the same wire pipe fastener component may be used for different types of endoscopes, e.g. both bronchoscopes, cystoscopes and ureteroscopes, whereas the roller component often may need to be of different design as the requirements for bending performance between different procedures may be very different. Bending performance involves parameters such as maximum bending angle, length of bending section and the relationship between movement of bending lever and degrees of bending of the distal tip.

The slot 135 may be formed by two similar projections 134 or walls as shown in FIGS. 12-17. The slot may also be formed by two different walls, e.g., having different length of projection from the encasement 80. Also, the walls may differ in thickness and shape.

Reusing the wire pipe fastener 84 between different types of endoscopes, and also having the same point C of crossing steering wires, facilitates a simpler manufacturing and enables that the same manufacturing equipment may be applied for the different types of endoscopes. Having the same point C of crossing makes it possible to use the same equipment for fixation of the steering wire end to the steering wire itself, by crimping. The crimping tool may be in one position. The handle shells with wire pipe fastener, roller, steering wires and wire pipes, are mounted in fixtures with the correct tension of the steering wires adjusted. The fixtures with handle shells are moved consecutively to the crimping tool where the crimping is done for both steering wires.

The wire pipe fastener 84 has two channels 136, or grooves or passages, adapted for the wire pipes 130 to pass through and be fixedly connected to the wire pipe fastener 84, which again is in a fixed connection to the handle of the endoscope. Each channel 136 has a proximal opening 137 and a distal opening 138. Each channel 136 may have one center axis a, b extending along the length of the channel. When the axes a, b are extended proximal the wire pipe fastener 84, the two axes a, b may cross each other between the wire pipe fastener 84 and the roller 90. The angle α between the two axes a, b will depend on the diameter of the roller 90 and the distance between the roller and the wire pipe fastener 84. The angle α may be in the range 20°-50°, or in the range 30°-40°.

The fixed connection of the wire pipes 130 inside the channels may be provided by gluing. The glue could preferably have a viscosity so that it will not flow out of the channels e.g., through the openings 137, 138, before it is cured. A glue which can be hardened by application of UV light may be used.

The wire pipe fastener is preferably made from a polymer material, such as polycarbonate (PC) or MABS. Both materials may be transparent, which is an advantage if a glue to be hardened by UV light is applied. Other polymers can also be applied, e.g. ABS if the transparency is not needed. If a polymer is applied to which it is difficult to achieve good adherence of the glue, such as polypropylene, the channel may have a geometry such that the hardened glue forms an anchor. Both the shape of the proximal openings 137 and the presence and the shape of ribs 139 may be used for providing such an anchor effect. The solidified adhesive might not be bonded to the channel 136 but, due to the shape imposed by the ribs and the openings, the solidified adhesive will resist the force applied by the bending lever 12 onto the steering wires and the wire pipes. In one example, the openings are smaller than a cross-section of the channel, therefore the anchor is wider and/or taller than the openings, so even though it its bonded to the wire pipes, the anchor cannot move through the openings. The ribs are optionally provided to increase the anchor effect.

Press-fit of the wire pipes 130 may also be applied for fixation of these to the wire pipe fastener 84. In that case, the ribs 39 could be designed to exert a sufficient pressure on the wire pipes. This would imply that the channel 136 with the ribs should be designed for one specific outer diameter of the wire pipes. The advantage of the glued solution is that the same channel, with or without ribs, can be used for different outer diameters of wire pipes. Fixation by a pressure fit has the advantage that the gluing process is avoided. Opposing ribs may be alternated to provide a slightly tortuous path in which the wire pipes can be press-fit.

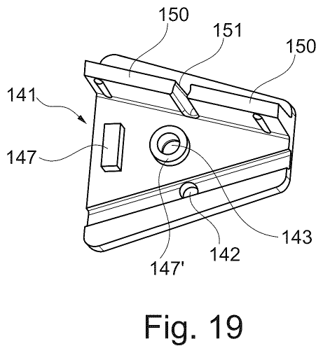

If a press-fit is applied for fixation of the wire pipes 130 in the wire pipe fastener 84, ribs 39 could be added also to the bottom of the two channels 136. Also, a cover could be placed on the top of the channels, e.g. as shown in FIG. 17. This cover could be provided with ribs extending into the channels for improving the press-fit of the wire pipes 130.

As shown, the channels are formed inside rectangular protrusions that protrude from the surface of the support platform of the encasement. The rectangular protrusions extend from a base 84′ of the wire pipe fastener 84. The base 84′ can protrude from a major surface of the encasement. Alternatively, the rectangular protrusions extend from the major surface, e.g. the support platform 80a, without the base 84′.

It is seen from FIGS. 12 to 14 that the bottoms of the two channels are not necessarily in the same plane or at the same level. In this example, if two planes are defined, one for each of the bottoms of the two channels, these planes could be parallel but may also be displaced or offset relative to each other. The displacement could be in the range 1-3 mm, preferably in the range 1.5-2.5 mm. A similar displacement may be made between the two fixing structures 132 for attachment of the steering wires 85 on the roller 90. However, the displacement between the points of fixating the two steering wires at the roller may not necessarily have the same distance as the displacement of the bottoms of the two channels but may preferably be within a similar size range.

This displacement has the advantage that the two steering wires will not be touching each other. This is important if the proximal ends of the wire pipes are placed distal to the point C of crossing, such that the friction of the steering wires are not increased. The displacement can also be an advantage in the arrangement of all the parts in the handle. For example, the encasement 80 shown in FIGS. 11 to 14 are provided with the protruding enclosure portion 82, providing more space inside the encasement. This more space may be used for the cable connecting the handle electronics to the monitor 15 and control unit 42. The extending part extends a portion of the lid away from the housing shell and elevates one of the channels relative to the housing shell. FIG. 15 shows a variation devoid of the protruding enclosure portion 82.

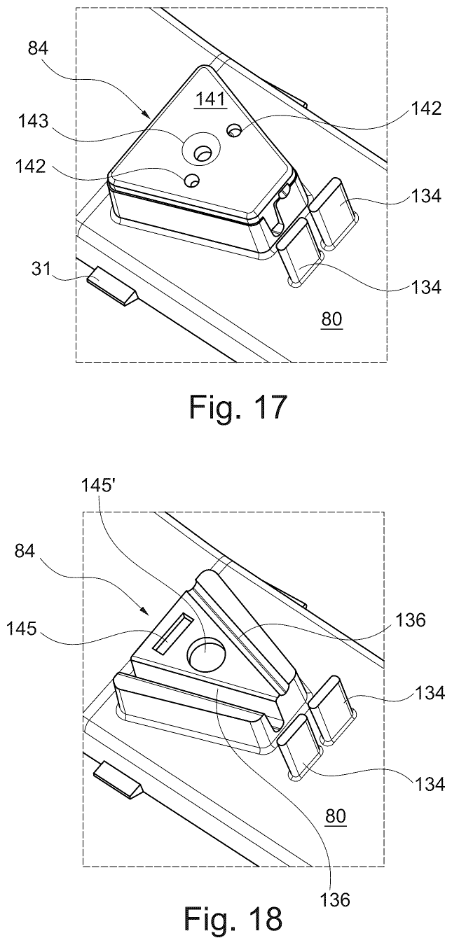

FIG. 16 shows the encasement 80 with the wire pipe fastener 84 arranged on a major wall part. This major wall part, or support platform, may be connected to the rest of the encasement by a glued sealing (not shown). Openings 140 for electrical cables are illustrated. The openings can be the same in all the variations of the encasement but they can be adapted to fit the cables of the camera of the endoscope. Press-fit and snap-fit parts and projections 134, forming the slot 135, are also shown. As stated above, the support platform 80a may also be formed in one piece with the circumferential walls and the one-piece part can be press-fit to the handle shell.

FIGS. 17 to 19 are views of a variation of the present embodiment in which a lid is adhesively bonded to form the channels. FIG. 17 shows a lid part 141 covering the channels 136. The lid part may be provided with glue holes 142 for application of glue for fixating the wire pipes 130 in the channels 136. Glue may also be applied through a glue hole 143 for securing the lid part 141 to the wire pipe fastener 84. The lid part 141 may be made from a transparent polymer to enable curing of the glue by UV light.

FIG. 18 shows the variation of FIG. 17 but with the lid part 141 removed. Here, the channels 136 are without ribs, but ribs may be provided if desired. It is seen that the wire pipe fastener 84 comprises indent areas 145, 145′ for receiving corresponding alignment taps 147, 147′ (see FIG. 19) on the lid part 141. As before, the channels are displaced relative to each other.

FIG. 19 shows the lid part 141, which in FIG. 17 is attached on the top of the wire pipe fastener 84 and closing the channels 136. FIG. 19 shows the side of the lid part 141 which faces the wire pipe fastener 84 when attached to it. The lid part 141 also comprises two extending wall parts 150 for entering the channel 136 in which the wire pipe 130 is intended to be placed closest to the encasement 80, in order to position the wire pipe 130. The two wall parts 150 are spaced apart and thereby form a passage 151 for glue between them. When the lid part 141 is arranged on the wire pipe fastener 84, the two wall parts 150 will extend into the channel 136 and glue can be applied into a glue hole and through the passage 151 for glue to contact the wire pipe 130. The walls 150 may be press-fit into one of the channels 136 to further secure the lid 141 to the wire pipe fastener 84.

FIGS. 20 to 22 illustrate a variation of the present embodiment where the wire pipe fastener 84 is arranged on a support platform 160, which is not part of a closed encasement 80. The support platform 160 may be connected to the housing shell 20′, e.g., by press-fit parts 41 or by a snap-fit connection. A printed circuit board (PCB) 170 may be separately connected to the housing shell 20′, e.g., by connecting means molded as part of the housing shell. The PCB 170 may be positioned between the housing shell 20′ and the support platform 160. This could also be by snap-fit or press-fit connections. Both the connections between the PCB 170 and the handle shell 20′, as well as the connections between the support platform 160 and the handle shell, may also be of other types, such as heat staking and ultrasound welding. There may be a mix of different types of connections applied.

The support platform 160, without the circumferential walls, is relevant when a sealed encapsulation of the PCB 170 is not necessary, e.g., for bronchoscopes, where a sealed encapsulation is not necessary. By avoiding a sealed encapsulation fewer components or parts may be used in the handle, and thereby the manufacturing costs may be reduced. The PCB 170 may be positioned between the housing shell 20′ and the support platform 160. This will provide some mechanical protection of the PCB during the rest of the assembly process. Also, this stacking of the PCB 170 and the wire pipe fastener 84 may utilize the space inside the handle in an optimal way.

As shown in FIG. 20, the projections 134 shown in FIGS. 9 to 18 are omitted. The steering wires are arranged to cross each other between the wire pipe fastener and the steering wire actuator/roller, also if the projections 134 are not present. However, the projections 134 may be provided. In some cases, the projections 134 facilitate assembly of the steering wires.

FIG. 21 shows that the wire pipe fastener 84 and the support platform 160 may be one integrated single part. This part may be molded as one part. It is seen that this integrated part may have support legs 162 for providing a correct positioning in relation to the handle shell 20′.

FIG. 22 shows the support platform 160 from the side opposite to the wire pipe fastener 84, this illustrates that the support platform 160 may be provided with a support platform rim 164 for stabilizing or stiffening the support platform 160. This is to prevent any minor movement of the wire pipe fastener 84 during manipulation of the bending lever 12 to bend the bending section 5. The stability of the position of the wire pipe fastener 84 influences the bending performance. An alternative to the support platform rim 164 would be a thicker support platform 160. But the application of the rim 164 will keep the amount of material applied, to obtain a given stiffness, at a minimum. The back side of the wire pipe fastener 84 can also be seen in FIG. 22 as a depression in the support platform 160.

FIG. 22 further shows support pins, or stand-offs, or component press-fit parts, 165, provided to secure the PCB 170. The support pins 165 extend from the support platform 160 and are designed to abut against the PCB when the endoscope handle 2 is assembled. This will stabilize the position of the PCB and reduce the risk of movements between the parts during use. The support pins 165 may be molded as an integrated part of the support platform 160.

In the different examples of the wire pipe fastener 84 shown and described above, different measures may be applied. In one example, the channels both have a width of approximately 2 mm, with around 1.5 mm at the bottom around 2.4 mm at the top of the channel. The length of each channel is in the range 13-19 mm. The distance between the two wire pipes 130 at their exit at the proximal end of the wire pipe fastener 84 may be 2-5 mm. The distance between the two wire pipes 130 at the distal entrance to the wire pipe fastener 84 may be 10-15 mm. The width of the slot opening 135 formed between the projections 134 may in an example be approximately 1-2 mm. In the example shown in FIG. 11, the distance between the point where a wire pipe exits the wire pipe fastener proximal end and the point at which the corresponding steering wire contacts the wire drum surface of the roller, is around 55-65 mm. In an example, the wire pipes extend proximally from the wire pipe fastener and pass the position C by a distance of 2-20 mm, preferably 4-15 mm.

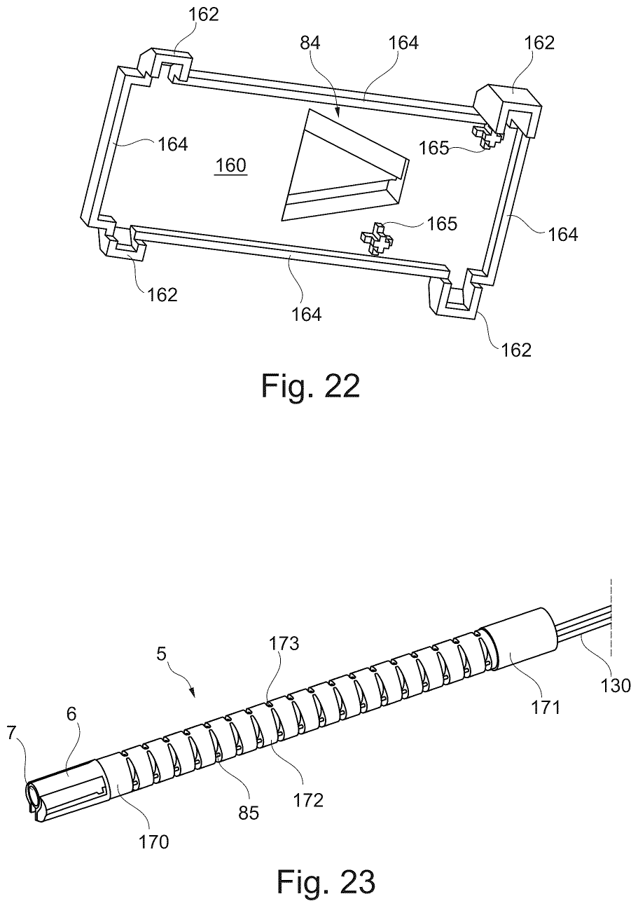

FIG. 23 shows a distal end of the insertion cord 3 of an endoscope, where the bending cover (not shown) typically covering the bending section 5 has been removed. The bending section is attached to the main tube 4 at the proximal end and to the distal tip 6 in the distal end. In this example, the bending section is molded in one piece comprising a number of segments including a distal end segment 170, a proximal end segment 171, and intermediate segments 172 therebetween. The proximal end segment 171 is connected to the distal end of the main tube 4. The segments are held together, or interconnected, by hinges 173, so that the segments can be bent relative to each other by manipulation of the steering wires 85. An example of such a bending section molded in one piece can be seen in commonly owned U.S. Pat. No. 10,321,804, which is incorporated herein by reference in its entirety.

Alternatively, the bending section 5 could be extruded in a relatively soft and resilient material, e.g., a foam-like material, with lumens for steering wires, electrical wires and the tubes passing through.

Steering wires 85 are connected in a fixed connection to the distal end, e.g., the distal end bending segment 170, i.e., the steering wire is preferably not movable in relation to the distal end bending segment. Between the handle and the distal end of the main tube 4, or the proximal end segment 171, the steering wires 85 are guided inside the wire pipes 130, which are secured in the handle 2 distally of the steering wire actuator 60. Other designs of the bending section are also possible.

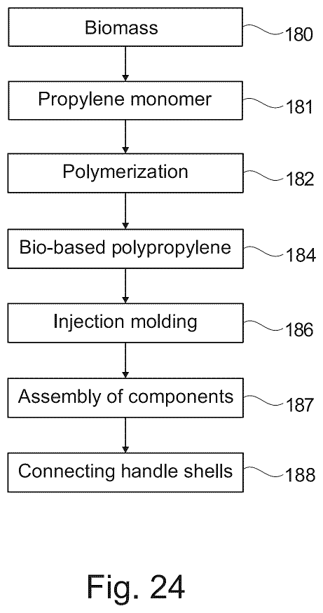

FIG. 24 shows a flowchart giving an overview of major steps in the process of providing and applying bio-based polypropylene for endoscope handles 2. A first step, 180, in this process, may be to provide the biomass needed to produce the bio-based PP.

At 181, propylene monomer is provided from the biomass. There are different methods for producing bio-based propylene, which is the starting monomer for PP. The PP is made from propylene by polymerization. The method selected for producing propylene may depend on the organic raw materials. For some raw materials, such as sugar and starch, fermentation may be a relevant step in providing the propylene. For some types of biomass, gasification may be a relevant step in providing the propylene. In general, gasification may be applied for a larger range of biomass sources, but fermentation is a cheaper and less energy consuming process.

At 182, propylene monomers are polymerized into polypropylene, e.g., by a chain-growth polymerization.

At 184, polymerization provides the bio-based polypropylene.

At 186, the bio-based polypropylene is applied for injection molding of the handle shells including the integrated fasteners and interfaces.

At 187, the different components, such as the encasing for the PCB, the wire pipe fastener, the roller, the suction valve, tubing for suction, buttons etc., are arranged in one housing shell and fastened by interfaces being integrated in the housing shell. Further, steering wires and wire pipes, as well as cables and the PCB may be arranged and the insertion cord and a working channel may be connected together.

Then, at 188, the two housing shells are connected, or clicked, together to form the housing of the handle.

The following items are further variations and examples of the embodiments described with reference to the figures.
1. An endoscope comprising: an insertion cord including a main tube and a bending section distal to the main tube; a handle comprising a steering wire actuator and a wire pipe fastener, the wire pipe fastener including a first and a second channel; a first steering wire and a second steering wire both connected to the steering wire actuator and running through the insertion cord so that manipulation of the steering wire actuator causes bending of the bending section; a first and a second wire pipe extending from the wire pipe fastener to a distal end of the main tube, the first and the second steering wire running inside the first and the second wire pipe, respectively; wherein the first and the second wire pipes are fixated in the first and the second channel of the wire pipe fastener, the first and the second wire pipe extending proximally of the wire pipe fastener along a first and a second axis, respectively, and wherein the first and the second axis cross each other in a position (C) between the wire pipe fastener and the steering wire actuator.
2. The endoscope according to item 1, wherein the first and the second channel each having a centerline extending in parallel with the first and second axis, respectively.
3. The endoscope according to any one of the previous items, wherein the endoscope comprising a support platform within the handle, the wire pipe fastener connected to and extending from the support platform, wherein the support platform and the wire pipe fastener are made in one piece.
4. The endoscope according to item 3, wherein the support platform is a wall part of an encasement for protecting a PCB.
5. The endoscope according to any one of the previous items, further comprising a structure defining a slot, wherein the slot is placed at the position (C) where the first and the second steering wires cross.
6. The endoscope according to any one of items 3-5, wherein the structure forming the slot projects from and is attached to the support platform.
7. The endoscope according to item 6, wherein the support platform, the wire pipe fastener, and the structure forming the slot, are made in one piece.
8. The endoscope according to item 5, wherein the structure forming the slot comprises two walls spaced apart by the slot.
9. The endoscope according to any one of the previous items, wherein the channels are arranged in parallel displaced planes.
10. The endoscope according to any one of the previous items, wherein the wire pipes extend proximally from the wire pipe fastener and passed the position (C), or through the slot.
11. The endoscope according to any one of the previous items, wherein a distance between the steering wire actuator and the position (C) is larger than a distance between the wire pipe fastener and the position (C), preferably the distance between the steering wire actuator and the position (C) is at least the double of the distance between the wire pipe fastener and the position (C).
12. The endoscope according to any one of the previous items, wherein the channels are defined by walls, where ribs extend from at least one of these walls, the ribs are preferably extending in a direction transverse to the direction of the first or second axis of the wire pipe placed in the respective channel.
13. The endoscope according to any one of the previous items, wherein the wire pipe fastener is provided with a lid part covering at least part of the first channel and of the second channel.
14. A method for assembling an endoscope according to any one of the previous items, comprising: providing a handle housing shell, arranging a wire pipe fastener and a roller in the handle housing shell, connecting a first steering wire and a second steering wire to the roller, connecting wire pipes, through which the steering wires are inserted, to the wire pipe fastener, adjusting the tension for each steering wire and following fixating the proximal end of the first steering wire to the first steering wire and fixating the proximal end of the second steering wire to the second steering wire.
15. An endoscope comprising a handle and an insertion cord with a bending section which is bendable by manipulation of steering wires movably arranged in wire pipes, the handle comprising: two shell parts assembled with fasteners, the fasteners made of the same material and in one-piece with one or another of the two shell parts, the assembled shell parts forming a compartment enclosed by the shell parts; at least one component selected from a group comprising: a roller for bending the bending section by pulling a steering wire, a wire pipe fastener connecting the wire pipes to one of the shell parts, an enclosure for a printed circuit board; and interfaces configured to secure the at least one component to at least one of the two shell parts inside the compartment, at least one of the interfaces made of the same material and in one-piece with the one or the other of the two shell parts, the interfaces extending into the compartment, the shell parts being made from polypropylene, wherein the polypropylene comprises bio-based polypropylene.
16. The endoscope of item 15, wherein the polypropylene comprises at least 20% bio-based polypropylene, alternatively, at least 40% bio-based polypropylene.
17. The endoscope according to any one of the preceding items, wherein the fasteners comprise snap fit fasteners or press fit fasteners.
18. The endoscope according to item 17, wherein 6-12 snap fits are applied to connect the two shell parts.
19. The endoscope according to any one of the preceding items, wherein both a roller for bending the bending section by pulling one of the steering wires, and a wire pipe fastener connecting the wire pipes to one of the shell parts, are secured to one of the shell parts inside the compartment via interfaces being integral with and made in the same material as a shell part.
20, The endoscope according to any one of the preceding items, wherein at least one further component selected from the group: a working channel port enabling insertion of tools into and through a working channel of the endoscope, a suction valve controlling suction through a working channel of the endoscope, are secured to at least one of the shell parts inside the compartment via interfaces being integral with and made in the same material as the at least one shell part.
21. The endoscope according to any one of the preceding items, wherein at least one interface is configured for heat staking or ultrasonic welding.
22. The endoscope according to any one of the preceding items, wherein the shell parts are connected to form the handle, and the shell parts are connected to each other by the fasteners, without any non-polypropylene material adding strength or stability to the connection.
23. The endoscope according to any one of the preceding items, wherein the components are connected to at least one of the shell part by the interfaces without any non-polypropylene material adding strength or stability to the connection.
25. The endoscope according to any one of the preceding items, wherein a roller is rotatably connected to a roller interface forming an axle for the roller, the roller interface being made of the same material and in one-piece with one of the shell parts, and ribs being provided to enforce a connection between the roller interface and the shell part.
26. The endoscope according to any one of items 15-25, further comprising the insertion cord including a main tube and a bending section distal to the main tube; a handle comprising a steering wire actuator and a wire pipe fastener, the wire pipe fastener including a first and a second channel; a first steering wire and a second steering wire both connected to the steering wire actuator and running through the insertion cord so that manipulation of the steering wire actuator causes bending of the bending section; a first and a second wire pipe extending from the wire pipe fastener to a distal end of the main tube, the first and the second steering wire running inside the first and the second wire pipe, respectively; wherein the first and the second wire pipes are fixated in the first and the second channel of the wire pipe fastener, the first and the second wire pipe extending proximally of the wire pipe fastener along a first and a second axis, respectively, and wherein the first and the second axis cross each other in a position (C) between the wire pipe fastener and the steering wire actuator.
27. The endoscope according to item 26, wherein the first and the second channel each having a centerline extending in parallel with the first and second axis, respectively.
28. The endoscope according to item 26, wherein the endoscope comprising a support platform within the handle, the wire pipe fastener connected to and extending from the support platform, wherein the support platform and the wire pipe fastener are made in one piece.
29. The endoscope according to item 28, wherein the support platform is a wall part of an encasement for protecting a PCB.
30. The endoscope according to items 26 to 29, further comprising a structure defining a slot, wherein the slot is placed at the position (C) where the first and the second steering wires cross.
31. Method of assembling an endoscope according to any one of the preceding items, the method comprising: providing a polypropylene material comprising at least 20% bio-based polypropylene; injection molding two shell parts from the polypropylene material, where the shell parts are molded with integrated fasteners configured to connecting the two shell parts to each other to form a handle of the endoscope, and, at least one of the shell parts are molded with at least one integrated interface configured to connect a shell part to at least one component selected from the group: a roller for bending the bending section by pulling a steering wire, a wire pipe fastener connecting the wire pipes to a handle shell, an enclosure for a printed circuit board; connecting the at least one component to a shell part by application of the at least one interface; and assembling the two shell parts by the fasteners thereby forming the handle having an internal compartment inside which the component is connected to a shell part.
32. A system comprising an endoscope according to any one of items 1-31, a monitor and a control unit.

We claim:

1. An endoscope comprising:
wire pipes;
steering wires arranged in the wire pipes;
an insertion cord including a bending section which is bendable by manipulation of the steering wires;
a working channel tube forming at least part of a working channel; and
a handle comprising:
housing shells including a first housing shell and a second housing shell, each of the first and the second housing shells comprising integral fasteners, the fasteners securing the first housing shell to the second housing shell to form an inner compartment, the fasteners and the housing shells comprising the same material and comprising snap-fit fasteners or press-fit fasteners;
a roller for bending the bending section by pulling the steering wires;
a suction valve configured to control suction through the working channel, the suction valve comprising a body and a stem translatable in the body to open or close the suction valve;
interfaces configured to secure the roller and the suction valve to at least one of the housing shells inside the inner compartment, at least one of the interfaces being integral with and comprising the same material as one or the other of the housing shells,
wherein at least 70% of the material of the housing shells comprises polypropylene material, and
wherein the body of the suction valve is comprised of at least 70% polypropylene material.

2. The endoscope of claim 1, wherein the polypropylene material comprises at least 20% bio-polypropylene.

3. The endoscope of claim 1, wherein the roller is rotatably connected to a roller interface forming an axle for the roller, the roller interface being integral and comprising the same material as one of the housing shells.

4. The endoscope of claim 3, wherein the roller interface comprises ribs provided to enforce a connection between the roller interface and the one of the housing shells.

5. An endoscope comprising:
steering wires arranged in wire pipes;
an insertion cord including a bending section which is bendable by manipulation of the steering wires; and
a handle comprising:
housing shells including a first housing shell and a second housing shell, each of the first and the second housing shells comprising integral fasteners and comprising at least 70% polypropylene material, the fasteners securing the first housing shell to the second housing shell to form an inner compartment, the fasteners and the housing shells comprising the same material;
at least one component selected from a group comprising: a roller for bending the bending section by pulling the steering wires, a wire pipe fastener connecting the wire pipes to one of the housing shell, and/or an encasement for a circuit board; and
interfaces configured to secure the at least one component to at least one of the housing shells inside the inner compartment, at least one of the interfaces being integral with and comprising the same material as one or the other of the housing shells, the interfaces extending into the inner compartment.

6. The endoscope of claim 5, wherein the polypropylene material comprises at least 20% bio-polypropylene.

7. The endoscope of claim 6, wherein the polypropylene material comprises at least 40% bio-polypropylene.

8. The endoscope of claim 5, wherein the fasteners comprise snap-fit fasteners or press-fit fasteners.

9. The endoscope of claim 8, wherein 6-12 snap-fits fasteners are used to attach the first housing shell to the second housing shell.

10. The endoscope of claim 9, wherein the housing shells further comprise integral guiding means.

11. The endoscope of claim 5, wherein the handle comprises the roller and the wire pipe fastener in the inner compartment.

12. The endoscope of claim 11, wherein the roller is rotatably connected to a roller interface forming an axle for the roller, the roller interface being integral and comprising the same material as one of the housing shells.

13. The endoscope of claim 12, wherein the roller interface comprises ribs provided to enforce a connection between the roller interface and the one of the housing shells.

14. The endoscope of claim 5, the endoscope further comprising a working channel tube forming at least part of a working channel, the handle further comprising a working channel port enabling insertion of tools into and through the working channel, and/or a suction valve to control suction through the working channel, wherein the working channel port and/or the suction valve are secured to at least one of the housing shells inside the inner compartment with the interfaces.

15. The endoscope of claim 5, the endoscope further comprising and a suction valve comprised of the at least 70% polypropylene material, the suction valve configured to control suction through the working channel, wherein the suction valve is secured to at least one of the housing shells inside the inner compartment with the interfaces.

16. The endoscope of claim 15, wherein the suction valve is connected to the at least one of the housing shells without any non-polypropylene material adding strength or stability to the connection.

17. The endoscope of claim 15, wherein the suction valve is connected to the at least one of the housing shells without any non-polypropylene material that is not integral with the at least one of the housing shells.

18. The endoscope of claim 5, wherein at least one of the interfaces is configured for heat staking or ultrasonic welding.

19. The endoscope of claim 5, wherein the first housing shell is secured to the second housing shell by the fasteners without any non-polypropylene material that is not integral with the at least one of the housing shells.

20. A system comprising the endoscope of claim 5, a monitor and a control unit.
